(19) United States
(12) Patent Application Publication
(10) Pub. No.: US 2023/0402159 A1
(43) Pub. Date: Dec. 14, 2023

(54) SYSTEM AND METHODS FOR PREDICTING PATIENT CARE PATHS

(71) Applicant: Change Healthcare Holdings, LLC, Nashville, TN (US)

(72) Inventor: Beata Konczewski, Rolling Meadows, IL (US)

(21) Appl. No.: 17/835,104

(22) Filed: Jun. 8, 2022

(51) Int. Cl. G16H 40/20; G06Q 10/10; G16H 40/67; G06Q 40/08

(52) U.S. Cl. CPC G16H 40/20; G06Q 10/10; G16H 40/67; G06Q 40/08

(57) ABSTRACT

A method of predicting a healthcare path for a patient includes receiving, from a remote device, eligibility data for the patient, the eligibility data associated with an eligibility check for a first visit between the patient and a healthcare provider; retrieving medical claims data from a database comprising deidentified information for a plurality of historical medical claims; predicting the healthcare path for the patient by aggregating the eligibility data with the medical claims data, wherein the healthcare path comprises one or more predicted subsequent visits and a predicted amount of time until each of the one or more subsequent visits; and transmitting, to the remote device, an alert comprising the predicted healthcare path.

### BACKGROUND

The present disclosure relates generally to healthcare data processing systems and, more specifically, to healthcare eligibility checks. An eligibility check is a way for a medical provider (e.g., a doctor, a hospital, etc.) to ensure that a patient is insured, as well as to confirm the patient's benefits, determine whether the provider is in the patient's network, determine prior authorization requirements, etc. For example, medical providers use patient data, such as insurance identifiers, patient identifiers, visit information, etc., to determine eligibility parameters, which in turn are used to determine if the patient can be treated, if a procedure is covered, patient copays, etc. Accordingly, eligibility checks often serve as an “entry point” into the healthcare revenue cycle for a patient and are often performed prior to an office visit. Often, third-party service providers handle eligibility checks based on data provided by medical providers, which reduces the on-site computational requirements for the medical providers.

### SUMMARY

One implementation of the present disclosure is a method of predicting a healthcare path for a patient. The method includes receiving, from a remote device, eligibility data for the patient, the eligibility data associated with an eligibility check for a first visit between the patient and a healthcare provider; retrieving medical claims data from a database comprising deidentified information for a plurality of historical medical claims; predicting the healthcare path for the patient by aggregating the eligibility data with the medical claims data, wherein the healthcare path comprises one or more predicted subsequent visits and a predicted amount of time until each of the one or more subsequent visits; and transmitting, to the remote device, an alert comprising the predicted healthcare path.

In some embodiments, the method further includes automatically scheduling at least one of the one or more subsequent visits.

In some embodiments, the alert causes the remote device to generate and display a graphical user interface that includes the healthcare path.

In some embodiments, transmitting the alert includes generating and transmitting one of an email, a text message, an automated phone call, a push notification, or graphical user interface data to the remote device.

In some embodiments, the alert is transmitted as a flat file extract.

In some embodiments, predicting the healthcare path further comprises providing the aggregated eligibility data and medical claims data to a predictive model, and wherein the predictive model outputs the prediction of the healthcare path.

In some embodiments, the eligibility data is received in an encrypted format, the method further comprising decrypting the eligibility data prior to predicting the healthcare path.

In some embodiments, the method further includes receiving, from the remote device, second medical claim data associated with the patient for the first visit; generating a hash of the second medical claim data; and storing the hash of the second medical claim data in the database.

In some embodiments, the eligibility data comprises one or more of a patient identifier, a provider identifier, a provider specialty, a date of the eligibility check, and a date of the first visit.

Another implementation of the present disclosure is a healthcare data processing system having a processor and memory having instructions stored thereon that, when executed by the processor, cause the processor to perform operations including receiving, from a remote device, eligibility data for the patient, the eligibility data associated with an eligibility check for a first visit between the patient and a healthcare provider; retrieving medical claims data from a database comprising deidentified information for a plurality of historical medical claims; predicting the healthcare path for the patient by aggregating the eligibility data with the medical claims data, wherein the healthcare path comprises one or more predicted subsequent visits and a predicted amount of time until each of the one or more subsequent visits; and transmitting, to the remote device, an alert comprising the predicted healthcare path.

In some embodiments, the operations further include automatically scheduling at least one of the one or more subsequent visits.

In some embodiments, the alert causes the remote device to generate and display a graphical user interface that includes the healthcare path.

In some embodiments, transmitting the alert includes generating and transmitting one of an email, a text message, an automated phone call, a push notification, or graphical user interface data to the remote device.

In some embodiments, the alert is transmitted as a flat file extract.

In some embodiments, predicting the healthcare path further comprises providing the aggregated eligibility data and medical claims data to a predictive model, and wherein the predictive model outputs the prediction of the healthcare path.

In some embodiments, the eligibility data is received in an encrypted format, the method further comprising decrypting the eligibility data prior to predicting the healthcare path.

In some embodiments, the operations further include receiving, from the remote device, second medical claim data associated with the patient for the first visit; generating a hash of the second medical claim data; and storing the hash of the second medical claim data in the database.

In some embodiments, the eligibility data comprises one or more of a patient identifier, a provider identifier, a provider specialty, a date of the eligibility check, and a date of the first visit.

Yet another implementation of the present disclosure is a non-transitory computer readable medium having instructions stored thereon that, when executed by one or more processors, cause the one or more processors to perform operations including receiving, from a remote device, eligibility data for the patient, the eligibility data associated with an eligibility check for a first visit between the patient and a healthcare provider; retrieving medical claims data from a database comprising deidentified information for a plurality of historical medical claims; predicting the healthcare path for the patient by aggregating the eligibility data with the medical claims data, wherein the healthcare path comprises one or more predicted subsequent visits and a predicted amount of time until each of the one or more subsequent visits; and transmitting, to the remote device, an alert comprising the predicted healthcare path.

In some embodiments, the operations further include automatically scheduling at least one of the one or more subsequent visits.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIGS. 1A and 1B are block diagrams of various communication architectures for predicting a patient's care path, according to some embodiments.

FIG. 2 is a detailed block diagram of a service provider computing device, according to some embodiments.

FIG. 3 is a flow diagram of a process for predicting a care path, according to some embodiments.

### DETAILED DESCRIPTION

Referring generally to the figures, a system and methods for predicting care paths for a patient are shown, according to various embodiments. A “care path” generally refers to a sequence of office visits, procedures, etc., for a patient receiving health care (i.e., entering the healthcare revenue cycle). In other words, a care path is a patient's journey through a healthcare system. For example, a care path may include a first (i.e., initial) visit to a first healthcare provider and any subsequent visits to the first healthcare provider or additional healthcare providers. A care path is generally determined based on the patient's first visit to a healthcare provider (e.g., for a particular ailment or procedure). For example, the care path for a patient visiting a cardiologist about a heart condition may include subsequent visits to the cardiologist, tests (e.g., a stress test, an electrocardiogram (ECG), etc.), procedures (e.g., heart surgery), etc. Accordingly, care paths may vary significantly between different patients and even an established care path may be variable. For example, parameters (e.g., provider specialty, urgency, etc.) or results of a first visit may determine or impact the type, timing, location, etc., of a subsequent visit.

As mentioned above, an eligibility check is often performed as a first step to a patient entering the healthcare revenue cycle (e.g., prior to the patient entering a care path) or as a first “step” in the patient's care path. The eligibility check can confirm the patient's insurance benefits, determine whether the provider is in the patient's network, determine prior authorization requirements, and more. Typically, a healthcare provider collects patient eligibility data, including the patient's name, date of birth, gender, ethnicity, address, insurance information (e.g., member and/or group number), etc., and either provides the eligibility data to a third-party service or processes the eligibility data on their own to determine the patient's eligibility. Traditionally, eligibility checks are performed prior to each patient visit or procedure; however, it would be beneficial to healthcare providers and payors to predict future visits and procedures from a single eligibility check.

Accordingly, the system and methods described herein predict a patient's care path based on eligibility data and historical health claims data. In this manner, the healthcare provider or payor can predict future visits and procedures for the patient to ensure that the patient will remain covered and to optimize patient care. For example, a predicted care path can allow a healthcare provider to quickly identify and better prioritize individuals who entered a complex or intense care path for targeted outreach, resulting in better clinical and financial outcomes. In some embodiments, alerts can be generated and transmitted to healthcare providers and/or payors based on the predicted care path for a patient. In some embodiments, subsequent visits and procedures can be automatically scheduled based on the predicted care path.

A key advantage to the system and methods described herein, as will be made clearer with respect to the description below, is the unique combination of historical electronic health claims data and eligibility data in developing a predicted care path. Specifically, the system described herein includes or references as massive, constantly updating database of claims data for a plurality of patients. Put another way, the historical electronic health claims data is robust and evolving, yielding highly accurate care path predictions. Further, care path predictions can be quickly generated for a large number of patients. These and other advantages are discussed in greater detail below.

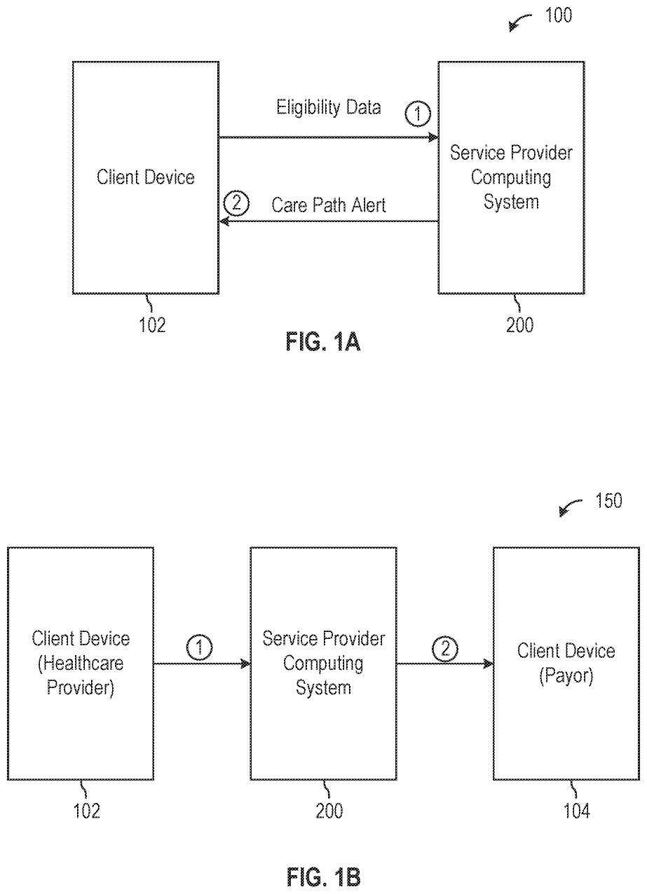

Referring generally to FIGS. 1A and 1B, block diagrams of various communication architectures for predicting a patient's care path are shown, according to some embodiments. Turning first to FIG. 1A, a first communication architecture 100 is shown to include a client device 102 in communication with a service provider computing device 200. As described herein, system 200 is generally a computing device or group of computing devices operated by a service provider; in particular, a service provider that analyzes and processes healthcare data. For example, system 200 may be operated by a healthcare revenue and/or payment cycle management service provider, who connects payors, healthcare providers, and/or patients. In some embodiments, system 200 is operated by a clearinghouse. Client device 102 may, accordingly, be any computing device operated by a client of the service provider. For example, client device 102 may be operated by a healthcare provider.

As shown, client device 102 and system 200 cooperatively send and receive (i.e., communicate) data relating a patient's eligibility and care path. In some embodiments, as discussed in greater detail below, client device 102 and system 200 communicate directly via a wired or wireless connection. In other embodiments, client device 102 and system 200 communicate indirectly, such as via a network. In other embodiments, while not shown in FIG. 1A or 1B, system 200 may intercept communications between client device 102 and another computing device. For example, client device 102 may transmit eligibility data to a third-party (e.g., other than system 200) for authorization and system 200 may intercept the eligibility data to predict a patient's care path, as discussed below. However, in general, system 200 may receive data directly from client device 102. It should also be appreciated that, while only a single client device is shown (e.g., client device 102), system 200 is generally configured to communicate with a plurality of client devices 102.

In some embodiments, client device 102 first transmits eligibility data (1) for a patient to system 200. Advantageously, the electronic communications between system 200 and client device 102 are encrypted to protect patient confidentiality. In some embodiments, the eligibility data is transmitted as an EDI 270/271 electronic eligibility and benefit inquiry. As described herein, eligibility data may include any of the patient's name, the patient's date of birth, a patient insurance identifier (e.g., insurance member ID, member subscriber number, group number, insurance company information), and information relating to the patient's visit (e.g., a type of visit, a procedure code or codes, a specialty identifier for the healthcare provider/doctor, etc.). Additionally, the eligibility data typically includes a date that the eligibility check was initiated and a date of the patient's visit or, put another way, a date that the visit is scheduled to occur (e.g., if the eligibility check is performed prior to the visit). In some embodiments, the eligibility data includes a provider identifier (e.g., national provider identifier (NPI)), encrypted patient information (e.g., a hash of the patient's name, date of birth, social security number, etc.), and the date of the eligibility check,

In response to receiving the eligibility data, system 200 may predict the patient's care path and may transmit, back to client device 102, an alert (2) based on the care path. In some embodiments, the alert is simply an indication (e.g., a report) of the predict care path. In some embodiments, the alert identifies one or more predicted future visits or procedures. In some embodiments, the alert causes client device 102 to display a user interface or otherwise convey the care path to a user. In some embodiments, the alert causes one or more predicted future visits/procedures to be scheduled.

Turning now to FIG. 1B, an alternate communication architecture 150 is shown in which system 200 transmits the alert (2) to a second client device 104 that is different from client device 102. Client device 104 may be operated by, for example, a payor (e.g., a health insurance company). In this manner, the operator of client device 104 may directly receive and review the patient's predicted care path. Where client device 104 is operated by a healthcare insurance company, for example, the healthcare insurance company can use the predicted care path for the patient to evaluate, pre-approve, and/or schedule subsequent visits/procedures. It will be appreciated that, for the sake of simplicity, system 200 is described in detail below with respect to only client device 102; however, it should be understood that system 200 can also communicate with client device 104 or other remote devices in a similar manner.

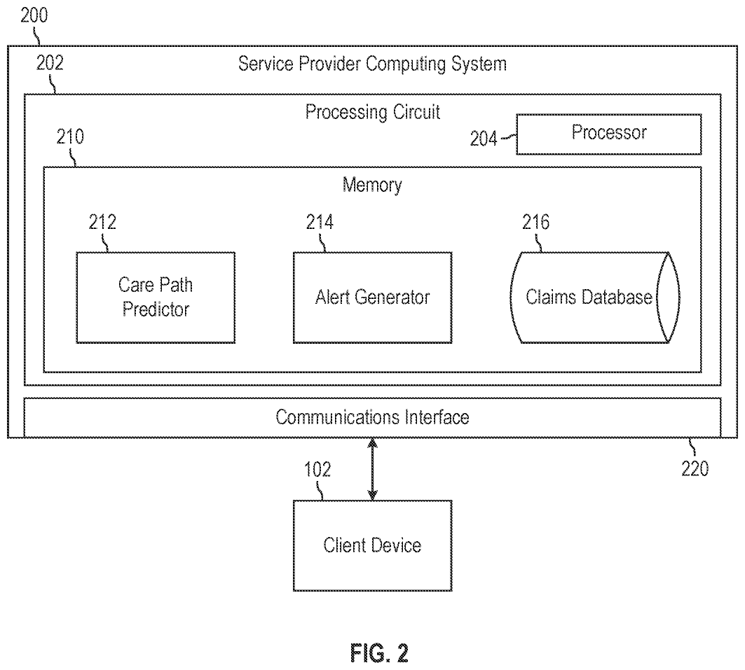

Referring now to FIG. 2, a detailed block diagram of system 200 is shown, according to some embodiments. As described above, system 200 generally include a single or multiple computing devices operated by a healthcare technology service provider. As shown, system 200 includes a processing circuit 202 that includes a processor 204 and a memory 210. Processor 204 can be a general-purpose processor, an application specific integrated circuit (ASIC), one or more field programmable gate arrays (FPGAs), a group of processing components, or other suitable electronic processing components. In some embodiments, processor 204 is configured to execute program code stored on memory 210 to cause system 200 to perform one or more operations. Memory 210 can include one or more devices (e.g., memory units, memory devices, storage devices, etc.) for storing data and/or computer code for completing and/or facilitating the various processes described in the present disclosure.

In some embodiments, memory 210 includes tangible, computer-readable media that stores code or instructions executable by processor 204. Tangible, computer-readable media refers to any media that is capable of providing data that causes system 200 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 210 can include random access memory (RAM), read-only memory (ROM), hard drive storage, temporary storage, non-volatile memory, flash memory, optical memory, or any other suitable memory for storing software objects and/or computer instructions. Memory 210 can include database components, object code components, script components, or any other type of information structure for supporting the various activities and information structures described in the present disclosure. Memory 210 can be communicably connected to processor 204, such as via processing circuit 202, and can include computer code for executing (e.g., by processor 204) one or more processes described herein.

While shown as individual components, it will be appreciated that processor 204 and/or memory 210 can be implemented using a variety of different types and quantities of processors and memory. For example, processor 204 may represent a single processing device or multiple processing devices. Similarly, memory 210 may represent a single memory device or multiple memory devices. Additionally, in some embodiments, system 200 may be implemented within a single computing device (e.g., one server, one housing, etc.). In other embodiments, system 200 may be distributed across multiple servers or computers (e.g., that can exist in distributed locations). For example, system 200 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. For example, virtualization software may be employed by system 200 to provide the functionality of a number of servers that is not directly bound to the number of computers in system 200.

Memory 210 is shown to include a care path predictor 212 configured to generate predicted patient care paths. At a high level, care path predictor 212 receives eligibility data for a patient from client device 102 (e.g., via a communications interface 220, described below) and aggregates the eligibility data with historical healthcare claims data (i.e., healthcare claims) retrieved from a claims database 216. In other words, care path predictor 212 both collects eligibility data from client device 102 and retrieves stored healthcare claims data, which is then combined (i.e., merged), to generate one or more predicted care paths. As described above, eligibility data may include patient data (e.g., including patient identifiers and patient insurance information) and information relating to the patient's visit (e.g., visit type, a procedure code or codes, a specialty identifier for the healthcare provider/doctor, date of visit, data of eligibility check, a service type code, etc.). In some embodiments, the eligibility data includes at least a patient identifier (e.g., a patient name), the patient's insurance information (e.g., a policy and/or group number), and a visit/procedure code or other indication of the healthcare provider's specialty. Specifically, in some embodiments, the eligibility data includes the provider's NPI, which is a HIPAA administrative simplification standard for identifying the provider.

Broadly speaking, claims database 216 maintains (e.g., receives, stores, and retrieves) electronic healthcare claim submissions (i.e., healthcare claims). For example, system 200 may receive or intercept electronic healthcare claims which are then stored in claims database 216. Generally, received electronic healthcare claims are deidentified or otherwise stripped of any personal identifying information (PII) associated with a patient to protect patient confidentiality. Specifically, the electronic healthcare claims and, in general, any information that is utilized by system 200, is maintained in compliance with HIPAA standards. Accordingly, in some embodiment, system 200 may generate a hash of a patient identifier when each new electronic healthcare claim is received and may store each new electronic healthcare claim with the generated hash. In this manner, only encrypted (e.g., hashed) data is stored in claims database 216. In some embodiments, electronic healthcare claims are submitted by client device 102, and therefore received by system 200, as part of an EDI 837 electronic claim transaction. It should also be noted that, in some embodiments, the eligibility data received by care path predictor 212 is encrypted to protect patient confidentiality. For example, the eligibility data generally includes a hash of the patient's first and last names, social security number or other identifier, date of birth, and the like. Accordingly, in some embodiments, care path predictor 212 is configured to decrypt the eligibility data. In other embodiments, care path predictor 212 may perform the operations described herein using only the patient hash.

It should be appreciated that the healthcare claims data utilized by care path predictor 212 may include any number of electronic healthcare claims. For example, care path predictor 212 may retrieve data for only a few historical claims, or thousands of claims, which is one advantage of utilizing claims database 216 when predicting care paths; specifically, that claims database 216 can contain, and therefore care path predictor 212 can reference, thousands or millions of previously-submitted healthcare claims. Those in the art will appreciate that such a robust claims database 216 can therefore provide numerous advantages in determining accurate care paths, as detailed herein. For example, accurately predicting the time between visits based on historical data can require a large number of datapoints (i.e., historical claims), as there is inevitably variation in visit timing. Additionally, while shown as a local database in FIG. 2, it should be appreciated that claims database 216 may be a remote database (e.g., hosted/maintained by a remote device), such as a database maintained on a cloud server.

As mentioned above, a care path generated by care path predictor 212 generally indicates one or more predicted future visits between a patient and one or more healthcare providers. In some embodiments, a care path “starts” with an initial visit to a first healthcare provider (e.g., a first doctor). For example, prior to or during an initial visit (e.g., a consultation for a procedure, a routine checkup, etc.), an eligibility check for the patient is performed and care path predictor 212 receives or intercepts at least a portion of the eligibility check data (e.g., part of an 270/270 transaction) indicating the patient's insurance information, and one or both of the healthcare provider's specialty (e.g., determined by the provider's NPI) and a visit identifier (e.g., a billing code, a code indicating the type of visit, etc.). Accordingly, various parameters of the first visit are generally determined based on the eligibility check data, including the provider's specialty, a date of the visit, a visit type (e.g., general check-up, diagnosis, surgery, etc.), and the like. By combining these first visit parameters determined from the eligibility data with historical claims data, care path predictor 212 can predict whether a future visit or multiple visits are expected, a type of future visit(s), and a timing between the first visit and any future visit.

To predict the time between visits, care path predictor 212 may first retrieve healthcare claims data from claims database 216. Specifically, care path predictor 212 may query claims database 216 using one or more parameters determined from the eligibility data. In some embodiments, care path predictor 212 retrieves only claims data associated with certain parameters determined by the eligibility data, as discussed above. For example, claims data that is associated with a similar visit type, a similar provider specialty, a similar billing code, etc., as that identified by the eligibility data may be retrieved. In some embodiments, patient demographic information (e.g., age, location, gender, etc.) can also be considered (e.g., by care path predictor 212) when retrieving historical claims, such that any retrieved healthcare claims are associated with a similar demographic of patient, for more accurate care path prediction. In some embodiments, care path predictor 212 retrieves claims data associated with multiple different parameters of the eligibility data.

Using the retrieved historical healthcare claims data, which again is limited to healthcare claims that have at least one common characteristic with the eligibility data for a patient (e.g., provider specialty), care path predictor 212 may calculate an average or median time between past healthcare visits (e.g., from multiple patients) that share one or more characteristics, such as visit type, provider specialty, etc. For example, care path predictor 212 may predict that a patient scheduling or conducting a first visit with an oncologist is likely to undergo various tests (e.g., cancer screenings) or procedures (e.g., radiation therapy, chemotherapy, etc.) in the near future. Accordingly, care path predictor 212 may calculate an average time between initial visits with an oncologist and any subsequent visits (e.g., “between a first visit and a second visit, patients waited, on average, 21 days”) based on historical healthcare claims for patients that have seen an oncologist and subsequently required testing, procedures, etc. As mentioned above, in some embodiments, the predicted time between visits may be further refined by using additional parameters to narrow the group of healthcare claims that are retrieved from claims database 216. For example, claims may be filtered based on the patient's age, location, etc., for a more accurate timing prediction. Thus, care path predictor 212 may generate a care path by combining the predicted visits with predicted visit timing as determined using the historical healthcare claims data.

In some embodiments, care path predictor 212 utilizes machine learning methods or artificial intelligence to predict patient care paths. For example, care path predictor 212 may include a machine learning model (e.g., a neural network) that is trained using the historical claims data in claims database 216 to predict future visits and the timing between visits. Specifically, the machine learning model may be trained using supervised learning techniques to predict a number, type, timing, etc., of future visits for a patient. Example machine learning models implemented by care path predictor 212 can include, but are not limited to, long short-term memory networks, random forest, gradient boosting regressor, time delay, etc. In some embodiments, the machine learning model is fed eligibility data including, for example, the date of the eligibility check, the date of a first visit, the healthcare provider's specialty (e.g., a code), and can output a predicted number, type, and timing of future visits. For example, the machine learning model may output at least a predicted number of days until a subsequent visit, which can then be used to generate a care path.

Memory 210 is also shown to include an alert generator 214 that generates and transmits alerts based on the care paths predicted by care path predictor 212. In some embodiments, alert generator 214 is configured to generate a user interface that is displayed via a display screen of system 200 (not shown) or a display screen of client device 102 (not shown). The user interface may include graphics, text, and the like to indicate to a user (e.g., a healthcare provider) the patient's care plan. In some embodiments, alert generator 214 generates and transmits (e.g., to client device 102) a text message, an email, or a push notification. In some embodiments, alert generator 214 initiates an automated voice call. In some embodiments, alert generator 214 transmits the alert as a flat file extract. In any case, alert generator 214 may indicate to an end-user the predicted care path for a patient, to include at least the next visit and timing of the next visit.

In some embodiments, alert generator 214 is configured to automatically schedule one or more visits between the patient and one or more healthcare providers based on the predicted care path. For example, alert generator 214 may transmit alerts to each of one or more healthcare providers based on the patient's eligibility data and predicted care path. In this example, alert generator 214 may automatically identify available healthcare providers based on the predicted visits in the patient's care path and may transmit alerts to the identified healthcare providers. The identified healthcare providers may, for example, have a specialty that aligns with one or more predicted future visits for the patient and/or may have availability in their schedules. In some embodiments, alert generator 214 (e.g., more broadly, system 200) may interface with one or more remote systems associated with the healthcare providers to receive updated scheduling information. For example, alert generator 214 may communicate with remote devices (e.g., client device 102) to automatically schedule future appointments.

Communications interface 220 may facilitate communications between controller and any external components or devices. For example, communications interface 220 can provide means for transmitting data to, or receiving data from, one or more client devices (e.g., including client device 102). Accordingly, communications interface 220 can be or can include a wired or wireless communications interface (e.g., jacks, antennas, transmitters, receivers, transceivers, wire terminals, etc.) for conducting data communications. In various embodiments, communications via communications interface 220 may be direct (e.g., local wired or wireless communications) or via a network (e.g., a WAN, the Internet, a cellular network, etc.). For example, communications interface 220 can include a WiFi transceiver for communicating via a wireless communications network. In another example, communications interface 220 may include cellular or mobile phone communications transceivers. In yet another example, communications interface 220 may include a low-power or short-range wireless transceiver (e.g., Bluetooth®).

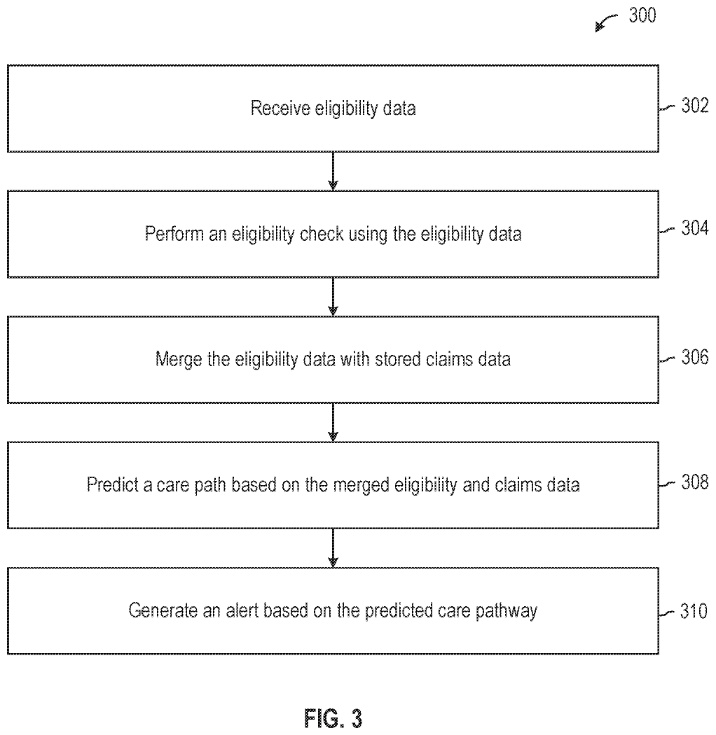

Referring now to FIG. 3, a flow diagram of a process 300 for predicting a care path is shown, according to some embodiments. As described above, predicted care path(s) (e.g., generated by process 300) may be customizable to each individual patient and, advantageously, may be predicted prior to, or at the same time as, a patient's first visit to a healthcare provider. Using the predicted healthcare path, healthcare providers and/or payors can coordinate to schedule appointments and payments to maximize the efficiency of patient care. In some embodiments, process 300 is implemented by system 200, as described above. It will be appreciated that certain steps of process 300 may be optional and, in some embodiments, process 300 may be implemented using less than all of the steps.

At step 302, eligibility data is received. In general, “eligibility data” is received as part of an eligibility check, which confirms a patient's health insurance coverage. Accordingly, the eligibility data received at step 302 generally includes at least an identifier for a patient (e.g., the patient's health insurance information), an identifier for the healthcare provider submitting the eligibility check (e.g., the healthcare provider's specialty), a date of a first visit associated with the eligibility check, and a date of the eligibility check. In some embodiments, the eligibility data includes the provider's NPI, hashed patient data (e.g., name, date of birth, SSN, etc.), and a date of the eligibility check. It will be appreciated that eligibility may include other information such as patient demographics, specific procedure codes, etc. As described above, in some embodiments, the eligibility data is received from a client device associated with (e.g., operated by) a healthcare provider. In some embodiments, the eligibility data is received as an EDI 270/271 electronic eligibility and benefit inquiry. In some embodiments, the eligibility data is encrypted prior to receipt and, accordingly, is decrypted at step 302. For example, in some embodiments, the patient information included in the eligibility data is hashed to protect the patient's privacy. Accordingly, in some embodiments, step 302 includes decrypting the patient data. However, in many cases, process 300 is performed using only hashed patient data, to avoid exposing and/or storing sensitive patient information.

At step 304, an eligibility check is optionally performed. Specifically, in some embodiments, the eligibility check is performed by system 200 based on the received eligibility data. For example, system 200 may query a database or a remote device (e.g., a server) associated with a healthcare insurance provider to check/confirm a patient's insurance coverages. In other embodiments, the eligibility check is performed by a system or device other than system 200. In such embodiments, system 200 merely intercepts eligibility data at step 302 and uses the intercepted eligibility data for the remaining steps of process 300. One of ordinary skill in the art will understand the process of performing an eligibility check; thus, the steps of performing an eligibility check are not described herein in detail for the sake of conciseness.

At step 306, the eligibility data is merged (i.e., aggregated) with stored claims data. As described above, “stored claims data” generally refers to a plurality of stored electronic healthcare claims (i.e., insurance claims) that are maintained by system 200 or another interconnected but remote system (e.g., a remote server). Advantageously, system 200 may maintain, or have access to, a robust database of historic (i.e., previously-submitted) electronic healthcare claims. To this point, accurate care path predictions are reliant on an accurate and robust historical data set. Each healthcare claim may include information such as a date of the associated visit or procedure, a healthcare provider identifier and specialty code, a reason for the visit or billing code, etc. In some embodiments, multiple historic claims are linked, mapped, or otherwise associated within a database. For example, multiple historic claims associated with the same patient may be linked. In another example, multiple historic claims associated with a similar type of procedure or provider specialty may be linked. However, it should be noted that the historic claims data is generally maintained with a hash of the associated patient's data (e.g., a hash of a patient identifier) to avoid storing patient PII. Accordingly, in some embodiments, system 200 may also (e.g., prior to or after process 300) receive or intercept electronic healthcare claims, generate a hash of the patient data for the healthcare claim, and store the encrypted healthcare claim data in a database.

In some embodiments, the historic healthcare claims data merged with the eligibility data is limited or filtered based, at least in part, on the eligibility data. For example, only historical healthcare claims that are associated with the same healthcare provider, healthcare provider specialty, geographic location, etc., may be merged. More generally, any parameter or set of parameters may be used to filter or otherwise limit the historical data that is merged with the eligibility data. As another example, eligibility data for a patient that is visiting a cardiologist may only be merged with historical healthcare claims for cardiologists. In some embodiments, additional parameters may further limit the merged data. To continue the previous example, the historical healthcare claims may be further limited to the specific cardiologist visited by the patient, the healthcare facility associated with the cardiologist, the patient's location, the patient's age, etc. In this manner, a more accurate care path can be predicted in subsequent steps of process 300, as only relevant data is being merged with the patient's eligibility data.

At step 308, a predicted care path is generated for the patient. As mentioned above, a care path generally indicates one or more predicted future visits between a patient and one or more healthcare providers. For example, a care path may “start” with an initial visit to a first healthcare provider (e.g., a first doctor), which may or may not be associated with the eligibility check performed at step 304, and may include subsequent visits to the first healthcare provider or other healthcare providers. In addition to indicating whether the patient is expected to require subsequent visits, the care path may indicate one or both of a type of subsequent visit and a timing of any subsequent visit. The visit “type” may define a provider specialty for the subsequent visit, a predicted reason for the subsequent visit, predicted billing codes for the subsequent visit, etc.

Visit timing generally refers to the amount of time between the first visit and one or more subsequent visits. In some embodiments, the amount of time between visits in the care path is predicted by calculating an average or median time between visits defined by the historical healthcare claims data. Put another way, each historical healthcare claims (e.g., that is merged with the eligibility data at step 306) may include a date on which the associated visit (e.g., between a patient and a healthcare provider) occurred. Using this information, the time between related visits can be calculated. For example, if a first visit for a first patient occurred on July 1^{st} and a second visit for the same patient occurred on July 21^{st}, and both visits were associated with a common healthcare provider or similar provider specialty, then the amount of time between visits is 20 days. It should be noted that, in some embodiments, a database or table is continuously or regularly updated as new healthcare claims are received to reflect the timing between visits. Once the amount of time between visits of each historical healthcare claims is determined, an average or median amount of time for a given healthcare provider specialty, visit type, billing code, or other parameter may be calculated. The average or median amount of time between similar visits to that associated with the eligibility data may then be utilized to generate the predicted care path. As mentioned above, in some embodiments, a machine learning model or artificial intelligence may be used to predict visit timing.

At step 310, an alert is generated back on the predicted care path. In some embodiments, the alert is generated as a user interface that is transmitted to, and/or displayed via a display screen of, a client device associated with the healthcare provider that initiated the eligibility check. The user interface may include graphics, text, and the like to indicate to a user (e.g., a healthcare provider) the patient's care plan. In some embodiments, the alert is a text message, an email, or a push notification. In some embodiments, the alert is an automated voice call. In some embodiments, the alert is generated and transmitted (e.g., to a client device) as a flat file extract. In any case, the alert generally indicates to an end-user the predicted care path for a patient, to include at least the next visit and timing of the next visit.

In some embodiments, step 310 further includes automatically scheduling one or more predicted future visits based on the predicted care path. For example, alerts transmitted to each of one or more healthcare providers based on the patient's eligibility data and predicted care path may prompt the healthcare provider(s) to contact the patient and initiate scheduling. In this example, healthcare providers can also be automatically identified based on the predicted visits in the patient's care path (e.g., based on visiting timing, location, type, etc.). The identified healthcare providers may, for example, have a specialty that aligns with one or more predicted future visits for the patient and/or may have availability in their schedules. In some embodiments, one or more remote systems associated with the healthcare providers may be accessed to receive updated scheduling information and/or to automatically schedule future appointments.

For additional clarity, an example use-case of process 300 is provided herein. In this example, a patient is establishing care for a knee replacement. Accordingly, as a first step in the healthcare process, the patient may call their healthcare provider (herein “Provider 1”) to schedule an initial appointment with a doctor. During or after the scheduling of the first appointment, Provider 1 may perform an eligibility check to confirm the patient's coverages, etc., by transmitting eligibility data (e.g., the patient's information, a date of the eligibility check, the provider's NPI, etc.) to a third-party or other remote system for checking eligibility. In some embodiments, the third-party is associated with client device 102, as described above. In other embodiments, client device 102 intercepts the eligibility data.

Once the client device receives the eligibility data (e.g., either directly or by intercepting the data), the client device determines the reason for the visit (e.g., knee replacement consultation) based on, for example, the Provider 1's NPI. For example, Provider 1's NPI may be used to determine that Provider 1 is an orthopedic surgeon. Additionally, or alternatively, other indicators (e.g., a billing or procedure code) may be included in the eligibility data that indicate the reasons for the visit. In any case, the client device may query a historical healthcare claims database to identify historical claims (i.e., electronic healthcare insurance claims) associated with Provider 1 and/or that match certain parameters of the eligibility data (e.g., patient's age, geographic location, visit/procedure type, etc.).

Once relevant historical claims data is identified, the relevant historical claims may be used to predict a care path. Specifically, in some embodiments, the time between visits for each historical claim may be determined and used to calculate an average time between visits. Additionally, the number and/or type of follow-up visits (e.g., surgery after the initial consultation, physical therapy, check-ups, etc.) can be predicted. Once the care path is predicted, an alert can be generated and sent back to Provider 1's computing system such that the initial and/or follow-up visits can be scheduled, preapproved, etc. Thus, process 300 improves the patient care and scheduling processes by accurately predicting a patient's care path even before the first visit.

### CONFIGURATION OF EXEMPLARY EMBODIMENTS

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

What is claimed is:

1. A method of predicting a healthcare path for a patient, the method comprising:
receiving, from a remote device, eligibility data for the patient, the eligibility data associated with an eligibility check for a first visit between the patient and a healthcare provider;
retrieving medical claims data from a database comprising information for a plurality of historical medical claims;
predicting the healthcare path for the patient by aggregating the eligibility data with the medical claims data, wherein the healthcare path comprises one or more predicted subsequent visits and a predicted amount of time until each of the one or more subsequent visits; and
transmitting, to the remote device, an alert comprising the predicted healthcare path.

2. The method of claim 1, further comprising automatically scheduling at least one of the one or more subsequent visits.

3. The method of claim 1, wherein the alert causes the remote device to generate and display a graphical user interface that includes the healthcare path.

4. The method of claim 1, wherein transmitting the alert comprises generating and transmitting one of an email, a text message, an automated phone call, a push notification, or graphical user interface data to the remote device.

5. The method of claim 1, wherein the alert is transmitted as a flat file extract.

6. The method of claim 1, wherein predicting the healthcare path further comprises providing the aggregated eligibility data and medical claims data to a predictive model, and wherein the predictive model outputs the prediction of the healthcare path.

7. The method of claim 1, wherein the eligibility data is received in an encrypted format, the method further comprising decrypting the eligibility data prior to predicting the healthcare path.

8. The method of claim 1, further comprising:
receiving, from the remote device, second medical claim data associated with the patient for the first visit;
generating a hash of the second medical claim data; and
storing the hash of the second medical claim data in the database.

9. The method of claim 1, wherein the eligibility data comprises one or more of a patient identifier, a provider identifier, a provider specialty, a date of the eligibility check, and a date of the first visit.

10. A healthcare data processing system comprising:
a processor; and
memory having instructions stored thereon that, when executed by the processor, cause the processor to perform operations comprising:
receiving, from a remote device, eligibility data for the patient, the eligibility data associated with an eligibility check for a first visit between the patient and a healthcare provider;
retrieving medical claims data from a database comprising information for a plurality of historical medical claims;
predicting the healthcare path for the patient by aggregating the eligibility data with the medical claims data, wherein the healthcare path comprises one or more predicted subsequent visits and a predicted amount of time until each of the one or more subsequent visits; and
transmitting, to the remote device, an alert comprising the predicted healthcare path.

11. The system of claim 10, the operations further comprising automatically scheduling at least one of the one or more subsequent visits.

12. The system of claim 10, wherein the alert causes the remote device to generate and display a graphical user interface that includes the healthcare path.

13. The system of claim 10, wherein transmitting the alert comprises generating and transmitting one of an email, a text message, an automated phone call, a push notification, or graphical user interface data to the remote device.

14. The system of claim 10, wherein the alert is transmitted as a flat file extract.

15. The system of claim 10, wherein predicting the healthcare path further comprises providing the aggregated eligibility data and medical claims data to a predictive model, and wherein the predictive model outputs the prediction of the healthcare path.

16. The system of claim 10, wherein the eligibility data is received in an encrypted format, the method further comprising decrypting the eligibility data prior to predicting the healthcare path.

17. The system of claim 10, the operations further comprising:
receiving, from the remote device, second medical claim data associated with the patient for the first visit;
generating a hash of the second medical claim data; and
storing the hash of the second medical claim data in the database.

18. The system of claim 10, wherein the eligibility data comprises one or more of a patient identifier, a provider identifier, a provider specialty, a date of the eligibility check, and a date of the first visit.

19. A non-transitory computer readable medium having instructions stored thereon that, when executed by one or more processors, cause the one or more processors to perform operations comprising:
receiving, from a remote device, eligibility data for the patient, the eligibility data associated with an eligibility check for a first visit between the patient and a healthcare provider;
retrieving medical claims data from a database comprising information for a plurality of historical medical claims;
predicting the healthcare path for the patient by aggregating the eligibility data with the medical claims data, wherein the healthcare path comprises one or more predicted subsequent visits and a predicted amount of time until each of the one or more subsequent visits; and
transmitting, to the remote device, an alert comprising the predicted healthcare path.

20. The computer readable medium of claim 19, the operations further comprising automatically scheduling at least one of the one or more subsequent visits.
